(19) United States
(12) Plant Patent Application Publication
(10) Pub. No.: US 2002/0100096 P1
(43) Pub. Date: Jul. 25, 2002

(54) CHRYSANTHEMUM PLANT NAMED 'MIDAS TIME GOLD'

(76) Inventor: Mark Roland Boeder, West Wittering (GB)

Correspondence Address: Mark K. Dickson, HOWREY SIMON ARNOLD & WHITE, LLP, 750 Bering Drive, Houston, TX, 77057-2198, US

(21) Appl. No.: 09/768,038

(22) Filed: Jan. 24, 2001

(51) Int. Cl. A01H 5/00

(52) U.S. Cl. PLT/295

(57) ABSTRACT

A new variety of Chrysanthemum plant named ‘Midas Time Gold,’ having golden-yellow quilled/spoon shaped ray florets with a dark green flower center.

### LATIN NAME OF THE GENUS AND SPECIES

Botanical classification: Dendranthema grandiflora.

### VARIETY DENOMINATION

The new Chrysanthemum variety denomination is ‘Midas Time Gold.’

### BACKGROUND OF THE INVENTION

The present invention comprises a new and distinct cultivar of Chrysanthemum botanically known as Dendranthema grandiflora, and referred to by the cultivar name ‘Midas Time Gold.’

‘Midas Time Gold,’ identified as MTG1, originated from a spontaneous whole plant mutation found by Mark Boeder in a controlled cultivated planting in Chichester, West Sussex, United Kingdom of the variety known as ‘Midas Time Yellow.’ The parent plant, ‘Midas Time Yellow,’ is an undistributed, unpatented variety. The new variety ‘Midas Time Gold’ has been asexually reproduced by vegetative stemcuttings in Leythorne Nursery, Chichester, West Sussex, United Kingdom and the distinguishing characteristics are retained through successive generations of asexual reproduction.

### BRIEF SUMMARY OF THE INVENTION

‘Midas Time Gold’ is a pot type of Chrysanthemum plant variety having golden-yellow ray florets with quilled and spoon shaped tips and a single flower type.

### Comparison with Parent

Plants of the new Chrysanthemum variety ‘Midas Time Gold’ are similar to the parent cultivar ‘Midas Time Yellow’ in plant habitat and growth rate. In side-by-side comparisons in Chichester, West Sussex, United Kingdom, under commercial practice, plants of the new Chrysanthemum variety ‘Midas Time Gold’ produced golden-yellow ray florets whereas the parent cultivar ‘Midas Time Yellow’ produced yellow flowers.

### Comparison with Other Varieties

Plants of the new Chrysanthemum variety ‘Midas Time Gold’ are dissimilar to the cultivar ‘Miramar,’ U.S. Plant Pat. No. 7,469, in plant habitat and growth rate. In side-by-side comparisons in Chichester, West Sussex, United Kingdom, under commercial practice, plants of the new Chrysanthemum variety ‘Midas Time Gold’ differed from plants of the cultivar ‘Miramar’ in at least the following characteristics:

1. The new variety ‘Midas Time Gold’ produced golden-yellow flowers whereas the cultivar ‘Miramar’ produced yellow flowers.

2. Plants of the new variety ‘Midas Time Gold’ have a smaller and shorter inflorescence than plants of the cultivar ‘Miramar.’

3. Plants of the new variety ‘Midas Time Gold’ have spoon/quill shaped ray florets whereas plants of the cultivar ‘Miramar’ have flat ray florets.

### BRIEF DESCRIPTION OF ILLUSTRATIONS

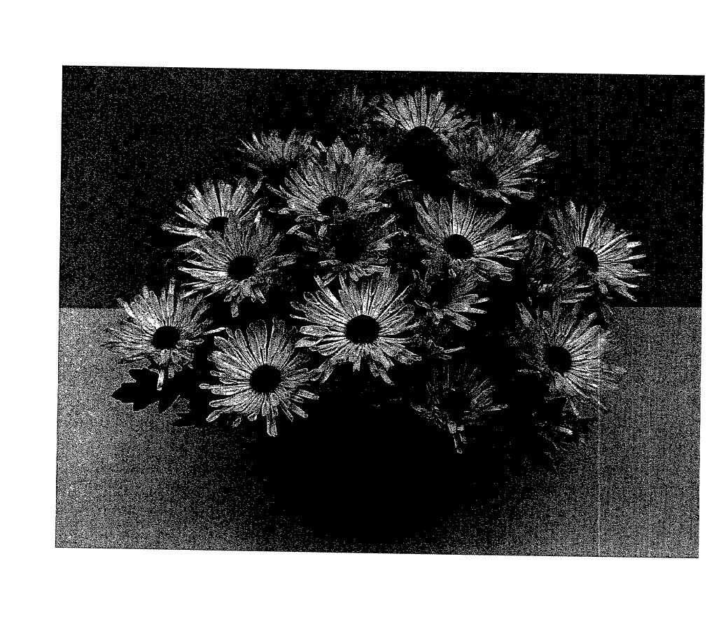

Typical specimens of the plant and flowers for the new Chrysanthemum variety ‘Midas Time Gold’ are shown in the accompanying scanned image printed from a digital photograph. The colors shown are as true as possible within the usual limits of this kind of illustration. FIG. 1 is a whole plant view of the new variety ‘Midas Time Gold’ grown in a pot.

### DETAILED BOTANICAL DESCRIPTION

The following description of the new Chrysanthemum variety ‘Midas Time Gold’ is of plants grown in a greenhouse at Leythorne Nursery, Chichester, United Kingdom in the month of November. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 18° C. at night and about 20° C. to 24° C. during the day under light conditions of about 5000 to 6000 foot candles. The plants respond well to the use of growth retardant, such as one B9 treatment at about 2 g/liter. To produce a commercial product the plants may be pinched once with the center bud removed. The typical container size for commercial growth is 1 liter. It has been observed that the shelf life of the new variety is about 21 days with a response time of about 7.5 weeks.

The new variety may be produced as a pot Chrysanthemum. The following description is with respect to a plant produced as a pot Chrysanthemum. In the description of this new Chrysanthemum variety, color values have been taken from The Royal Horticultural Society Colour Chart (R.H.S.C.C.).

Plant:

Plant type.—pot chrysanthemum.

Habit.—upright.

Height.—20-25 cm.

Branching characteristics.—upright spreading.

Length of Lateral Branches.—15-17 cm.

Number of Breaks from Pinch.—4-5.

Stem Color.—near 137C.

Response Time.—7.5 weeks.

Vigor.—short to very short.

Shelf Life.—near 21 days.

Disease Resistance.—no observations.

Growth retardant type and treatment.—1 application of 2 gram/liter B9, 21 days after planting.

Propagation.—Type: unrooted cuttings in pot under plastic sheet Time to rooting: 14 days with soil temperatures of 21° C. Rooting habit: evenly distributed over pot.

Foliage:

Number of Leaves Per Lateral Branch.—7-9.

Compound or single.—single.

Arrangement of Leaves.—alternate. Shape of Leaf: typical 5 lobed, ovate Size of Leaf: Width (cm): 0-5.0 Length (cm): 0-10.0 Leaf Apex: mucronate Base: cordate Attachment: petioled Aspect: slight undulating Margin: palmately lobed Surface characteristics: Top: pubescent Bottom: pubescent.

Petiole.—Color: near 137B Length: 0-2.5 cm.

Venation (describe).—1 prominent midvein, 4-6 well developed side veins Color: upper side: near 138C under side: near 138B.

Color.—Mature Leaf, upper side: near 139A under side: near 137C Young Leaf, upper side: near 139A; under side: near 137C.

Flower:

Flower appearance.—single flower with large golden-yellow ray flowers having a quill/spoon shaped tip and a dark green center.

Flower type.—single.

Flower shape.—flat.

Flowering habit.—top-down.

Number of blossoms per branch.—2-4.

Inflorescence form.—cyme Depth of fully expanded blossoms: 2.5-3.5 cm Diameter of fully expanded blossoms: 7.0-9.0 cm.

Sepals.—Number: 25-30 Color: near 137B Length: 0.6-1.1 cm Width: 0.2-0.6 cm Texture/Appearance: pubescent.

Peduncles are more or less equal in length over all flowers.—length is on average 5.5 cm. Length: 4.0-6.0 cm Color: near 138A Surface: pubescent Habit: slight undulating Strength: strong.

Pedicel.—Length: terminal: 14.0-15.0 cm; lateral: 15.0-16.0 cm Color: near 138A Surface: pubescent Habit: slight undulating Strength: strong.

Ray Florets.—Form/Shape: partly quilled/partly spoon shaped in 1 row Texture/Appearance: matte Number per flower: 50-60 Length: 2.5-4.0 cm Width: 0.3-0.6 cm Apex: rounded Base: tubular Margin: entire.

Disc florets.—Form/Shape: cylindrical Texture/Appearance: glossy Number per flower: 180-220 Length: 0.5-0.8 cm; Width 0.1-0.2 cm Diameter of disc: 1.8-2.3 cm.

Fragrance.—faint.

Flower Bud (at onset of color).—Length: 1.3-1.6 cm Diameter: 1.0-1.4 cm Form/shape: spherical.

General flower color.—1. Ray florets, upper side: Immature: near 21A Mature: near 23B Older/Fading: near 16B 2. Ray florets, under side: Immature: near 7A Mature: near 12B Older/Fading: near 12C 3. Disc florets: Immature: near 137B Mature: near 151B Older/Fading: near 151D 4. Bud: near 147A.

Flower progression with age.—dark green center disappears when flowers matures. Some pollen developing in center.

Reproductive organs:

Ray Florets.—Pistillate Pistil number: about 5 Stigma color: near 2A Stigma shape: forked Style Color: near 154B Style Length: 0.2-0.6 cm.

Disc Florets.—Pistillate Pistil Number: 5 Stigma color: near 2A Stigma shape: fork Style Color: near 154B Style Length: 0.2-0.4 cm. Staminate: Stamen Number: 5 Anther Shape: thread Anther Color: near 14B Pollen Color: near 13A Pollen Quantity: little.

What is claimed is:

1. A new and distinct variety of Chrysanthemum plant named ‘Midas Time Gold’ as described and illustrated.
